(19) United States
(12) Patent Application Publication
(10) Pub. No.: US 2023/0348935 A1
(43) Pub. Date: Nov. 2, 2023

(54) VACCINE COMPOSITIONS FOR MUCOSAL IMMUNE RESPONSE

(71) Applicants: NANT HOLDINGS IP, LLC, Culver City, CA (US); NANTCELL INC., Culver City, CA (US)

(72) Inventors: Patrick SOON-SHIONG, Culver City, CA (US); Elizabeth GABITZSCH, Culver City, CA (US); Victor PEYKOV, Culver City, CA (US)

(21) Appl. No.: 18/246,279

(22) PCT Filed: Oct. 22, 2021
(86) PCT No.: PCT/US2021/056184

§ 371 Date: Mar. 22, 2023

Related U.S. Application Data
(60) Provisional application No. 63/104,770, filed on Oct. 23, 2020.
(60) Provisional application No. 63/082,907, filed on Sep. 24, 2020.

(51) Int. Cl. A61K 9/19; C12N 15/86; A61K 39/215; C07K 14/165

(52) U.S. Cl. CPC C12N 15/86; A61K 9/19; A61K 39/215; C07K 14/165; C12N 2710/10343

(57) ABSTRACT

Vaccine compositions are provided that comprise a lyophilized, adenovirus-based expression vector, and a stabilizing compound, such as such as aragonite. Further provided are compositions that include a solid dosage form made from aragonite for loading and delivery of a vaccine composition.

### CROSS REFERENCE TO RELATED APPLICATIONS

This application claims the benefit of priority under 35 U.S.C. § 119(e) to U.S. Provisional Pat. Application Serial No. 63/104,770, filed Oct. 23, 2020 and to U.S. Provisional Pat. Application Serial No. 63/082,907, filed Sep. 24, 2020, each of which is incorporated herein by reference.

### FIELD

The present disclosure generally relates to the field of vaccine compositions. More specifically, the present disclosure relates to vaccine compositions having improved stability and ease of administration as well as to solid dosage forms of a vaccine for effective administration and manufacturing.

### REFERENCE TO A SEQUENCE LISTING

This application contains references to amino acid sequences and/or nucleic acid sequences which have been submitted concurrently herewith as the sequence listing text file “8774-16-PCT_Sequence_Listing_ST25.txt,” file size 30,000 Bytes (B), created on 22 Oct. 2021. The aforementioned sequence listing is hereby incorporated by reference in its entirety pursuant to 37 C.F.R. §1.52(e)(5).

### BACKGROUND

Vaccines can be made by letting cultures sit, thereby attenuating the virulence of the infectious organism. More recently, however, modern recombinant technologies have been used to produce antigenic proteins, and to produce viral vectors capable of expressing antigenic proteins against various infectious organisms.

Regardless of method or technology used to produce a vaccine, a persistent challenge has been the ability to store vaccines while retaining efficacy. Presently, an important part of maintaining vaccine efficacy relies on an expensive chain of cold storage systems. To mitigate the need for such expensive supply chains, manufacturers have tried adding chemical stabilizers to vaccines. However, such fillers often have toxicity and induce allergic reactions.

In addition to stability, vaccination protocols suffer from issues relating to routes of delivery. Many vaccines are administered through injection, which many people dislike.

Vaccines are traditionally delivered by intramuscular, intradermal, or subcutaneous injections. These injections can produce strong systemic immune responses, while the efficacy for triggering mucosal immune responses are variable and often weak or undetectable, particularly for subunit vaccines. From the draining lymph nodes that have processed the injected vaccine, antigen specific cytotoxic T cells (CTLs) and antibodies produced by B cells can migrate to different organs in the body but their migration to the various mucosal tissues (e.g., genital, intestinal, respiratory) is often limited or not possible due to inadequate homing mucosal receptors and chemotaxis. However, the intranasal route that is also considered as a parenteral immunization route can trigger good mucosal immune responses in the respiratory, genital and intestinal tract that are sharing some interconnections, which is more accessible if the vaccine is delivered at the mucosal site. Therefore, such parenteral vaccines may offer protection in some cases against mucosal pathogens.

Because most pathogens (e.g., COVID19) enter the body through mucosal tissues (oral, respiratory, genital, and interest enter the body through mucosal tissues (oral, respiratory, genital, and intestinal tracts) and many of them only replicate in the mucosal tissues, mucosal vaccination may optimally induce front line defense by inducing both innate (e.g., NK cells) and adaptive (T and B cells) immune responses at the local and distant mucosal sites.

Even in view of the currently approved vaccines for the Corona Virus Disease 2019 (COVID-19), vaccine distribution and administration on a rapid and global scale still poses hurdles. Global immunization has still not been achieved with the current approved vaccines due to many factors including manufacturing and/or storage costs and requirements.

Mucosal vaccine delivery (via the buccal, sublingual, nasal, oral, or vaginal mucosa) has received increasing interest as a means of inducing local and distant antibody immune response as well as systemic immune response. In addition, mucosal vaccine delivery by solid dosage forms (e.g., buccal/sublingual tablets, oral tablets or capsules, vaginal inserts) can offer several advantages such as the potential for mass immunization, patient compliance, ease of use, product shelf life stability, cold-chain independent capability. Furthermore, mucosal vaccine delivery can be suitable for patients that have needle injection phobia and the patient can self-administrate the vaccine with adequate explanations. The buccal/sublingual route has been used for many years to deliver drugs and small molecules to the bloodstream, but its application as a means of mucosal delivery for vaccines has not been developed as conventional.

Solid dosage forms including powders, tablets, or capsules for drug delivery require disintegration and release of the active ingredient (e.g., vaccine) at the time of administration. At the same time, the dosage form loaded with the active ingredient must be stable during transport and storage from the time of production up until administration. As such, use of a solid dosage form requires stability of the active ingredient loaded therein which then readily disintegrates in an aqueous solution (e.g., water, saliva, or mucosal fluid).

Thus, there is a need for a vaccine composition having improved stability, and an improved ease of administration, thereby enhancing vaccine coverage effectiveness. Further, there is a need for an improved solid vaccine dosage form that provides a compatible platform for a vaccine load, is stable prior to disintegration (administration), readily disintegrates, has low manufacturing cost, and facile administration for effective global immunization. The present disclosure addresses these needs and provides additional benefits as well.

### SUMMARY

One embodiment relates to a composition comprising a lyophilized adenovirus vector and a filler comprising a carbonite mineral, wherein the adenovirus vector comprises a nucleic acid molecule encoding at least a portion of a heterologous protein.

In one aspect, the adenovirus vector is derived from a type 5 adenovirus, and wherein the adenovirus has deletions in the E1, E2b, and E3 regions.

In various aspects, the heterologous protein comes from a virus.

In various aspects, the heterologous protein comes from a virus selected from the group consisting of SARS-CoV-2, MERS-CoV, SARS-CoV, HCoV-NL63, HCoV-229E, HCoV-OC43, HKU1, and influenza virus.

In various aspects, the heterologous protein comes from SARS-CoV-2. In some aspects, the heterologous protein is a spike (S) protein, a nucleocapsid (N) protein, or a membrane (M) protein. In yet another aspect, the heterologous protein is at least 80%, optionally at least 85%, optionally at least 90%, optionally at least 95%, optionally at least 97%, or even 100% identical to SEQ ID NO: 1, SEQ ID NO:2, SEQ ID NO:3, or SEQ ID NO:4

In various aspects, the adenovirus vector has between 0.5% and 5% residual moisture content. In one aspect, the adenovirus vector has less than 5% residual moisture. In yet another aspect, the adenovirus vector has less than 3% residual moisture.

In various aspects, the carbonite mineral has an orthorhombic lattice. In one aspect, the carbonite mineral is selected from the group consisting of aragonite, cerrusite, strontianite, witherite, and rutherfordine. In one aspect, the carbonite mineral is aragonite.

In various aspects, the filler comprises one or more compounds selected from the group consisting of lactose, sucrose, magnesium stearate, glucose, mannitol, sorbitol, starch, dextrose, maltodextrin, maltitol, and plant cellulose.

In various aspects, the filler lacks one or more compounds selected from the group consisting of lactose, sucrose, magnesium stearate, glucose, mannitol, sorbitol, starch, dextrose, maltodextrin, maltitol, and plant cellulose.

In various aspects, the composition comprises one or more compounds selected from the group consisting of sodium chloride, potassium chloride, sodium citrate, sodium phosphate, sucrose, dimethylglycine, glycine, methylsulfonylmethane, and yeast lysate.

Another embodiment relates to a capsule comprising the composition as disclosed herein. In one aspect, the capsule is enteric coated. In still another aspect, the capsule comprises alginate.

Another embodiment relates to a solid dosage form for delivery of a vaccine composition by oral, sublingual, or buccal administration of the vaccine composition, the solid dosage form comprising an aragonite composition comprising a plurality of aragonite particles, wherein the plurality of aragonite particles are impregnated with carbon dioxide (CO2); and a biocompatible polymer and a disintegrating agent mixed with the aragonite composition; wherein the solid dosage form further comprises the vaccine composition, and wherein the solid dosage form is a powder, a tablet, or a capsule.

In one aspect, the solid dosage form further comprises at least one excipient and is formulated to form a lozenge.

In another aspect of the solid dosage form, the plurality of aragonite particles have an average particle size of between 100 nm to 1 mm.

In various aspects of the solid dosage form, the biocompatible polymer is selected from polylactic acid (PLA), polyethylene, polystyrene, polyvinylchloride, polyamide 66 (nylon), polycaprolactame, polycaprolactone, acrylic polymers, acrylonitrile butadiene styrene, polybenzimidazole, polycarbonate, polyphenylene oxide/sulfide, polypopylene, teflon, polylactic acid, aliphatic polyester such as polyhydroxybutyrate, poly-3-hydroxybutyrate (P3HB), polyhydroxyvalerate, polyhydroxybutyrate-polyhydroxyvalerate copolymer, poly(3-hydroxybutyrate-co-3-hydroxyvalerate), polyglyconate, poly(dioxanone) and mixtures thereof.

In one aspect of the solid dosage form, the biocompatible polymer is PLA. In one aspect of the solid dosage form, the biocompatible polymer is Eudragit L30 D-55 (Evonik).

In various aspects of the solid dosage form, the disintegrating agent is selected from a starch, modified cellulose gums, insoluble cross-linked polyvinylpyrrolidones, starch glycolates, micro crystalline cellulose, pregelatinized starch, sodium carboxymethyl starch, low-substituted hydroxypropyl cellulose, homopolymers of N-vinyl-2-pyrrolidone, alkyl-, hydroxyalkyl-, carboxyalkyl-cellulose esters, alginates, and microcrystalline cellulose and its polymorphic forms.

In one aspect of the solid dosage form, the disintegrating agent is pea starch.

In one aspect of the solid dosage form, the vaccine composition comprises a recombinant viral expression construct encoding a viral protein or fragment thereof. In one aspect, the viral protein or fragment thereof corresponds to a coronavirus protein or fragment thereof. In still another aspect, the coronavirus protein or fragment thereof is a SARS-CoV2 virus binding protein. In one aspect, the recombinant ACE2 protein has at least 85% sequence identity to SEQ ID NO:5. In still another aspect, the recombinant ACE2 protein comprises the sequence of SEQ ID NO:6. In yet another aspect, the recombinant ACE2 protein comprises at least one mutation selected from T27F, T27W, T27Y, D30E, H34E, H34F, H34K, H34M, H34W, H34Y, D38E, D38M, D38W, Q24L, D30L, H34A, and D355L.

In one aspect of the solid dosage form, the vaccine composition comprises an adenovirus expression construct.

Another embodiment relates to a method of making a solid dosage form for loading a vaccine, the method comprising providing an aragonite composition comprising a plurality of aragonite particles impregnated with carbon dioxide (CO2); mixing the aragonite composition with a biocompatible polymer and a disintegrating agent to form the solid dosage form; and adding the vaccine composition to the solid dosage form.

In one aspect of the method of making a solid dosage form, the mixing of the aragonite composition with the biocompatible polymer and a disintegrating agent comprises hot melt extrusion.

In another aspect of the method of making a solid dosage form, the solid dosage form is a powder, a tablet, or a capsule.

In still another aspect of the method of making a solid dosage form, the solid dosage form is a tablet and wherein the method further comprises compacting the solid dosage form.

In another aspect of the method of making a solid dosage form, the aragonite composition and the biocompatible polymer are in a weight ratio of between 95:5 to 5 to 95.

In yet another aspect of the method of making a solid dosage form, the plurality of aragonite particles have an average particle size of between 100 nm to 1 mm.

In another aspect of the method of making a solid dosage form, the biocompatible polymer is selected from polylactic acid (PLA), polyethylene, polystyrene, polyvinylchloride, polyamide 66 (nylon), polycaprolactame, polycaprolactone, acrylic polymers, acrylonitrile butadiene styrene, polybenzimidazole, polycarbonate, polyphenylene oxide/sulfide, polypopylene, teflon, polylactic acid, aliphatic polyester such as polyhydroxybutyrate, poly-3-hydroxybutyrate (P3HB), polyhydroxyvalerate, polyhydroxybutyrate-polyhydroxyvalerate copolymer, poly(3-hydroxybutyrate-co-3-hydroxyvalerate), polyglyconate, poly(dioxanone) and mixtures thereof.

In still another aspect of the method of making a solid dosage form, the biocompatible polymer is PLA. In still another aspect of the solid dosage form, the biocompatible polymer is Eudragit L30 D-55 (Evonik).

In yet another aspect of the method of making a solid dosage form, the disintegrating agent is selected from a starch, modified cellulose gums, insoluble cross-linked polyvinylpyrrolidones, starch glycolates, micro crystalline cellulose, pregelatinized starch, sodium carboxymethyl starch, low-substituted hydroxypropyl cellulose, homopolymers of N-vinyl-2-pyrrolidone, alkyl-, hydroxyalkyl-, carboxyalkyl-cellulose esters, alginates, and microcrystalline cellulose and its polymorphic forms.

In still another aspect of the method of making a solid dosage form, the disintegrating agent is pea starch.

In one aspect of the method of making a solid dosage form, the vaccine composition comprises a recombinant viral expression construct encoding a viral protein or fragment thereof. In one aspect, the viral protein or fragment thereof corresponds to a coronavirus protein or fragment thereof. In still another aspect, the coronavirus protein or fragment thereof is a SARS-CoV2 virus binding protein. In one aspect, the recombinant ACE2 protein has at least 85% sequence identity to SEQ ID NO:5. In still another aspect, the recombinant ACE2 protein comprises the sequence of SEQ ID NO:6. In yet another aspect, the recombinant ACE2 protein comprises at least one mutation selected from T27F, T27W, T27Y, D30E, H34E, H34F, H34K, H34M, H34W, H34Y, D38E, D38M, D38W, Q24L, D30L, H34A, and D355L.

In one aspect of the method of making a solid dosage form, the vaccine composition comprises an adenovirus expression construct.

Embodiments discussed in the context of methods and/or compositions described herein may be employed with respect to any other method or composition described herein. Thus, an embodiment pertaining to one method or composition may be applied to other methods and compositions as well.

Other objects, features and advantages will become apparent from the following detailed description. It should be understood, however, that the detailed description and the specific examples, while indicating particular embodiments, are given by way of illustration only, since various changes and modifications within the spirit and scope of the invention will become apparent to those skilled in the art from this detailed description.

### BRIEF DESCRIPTION OF THE DRAWINGS

FIG. 1 shows the CaCO₃ chemical structures and symmetry for each of calcite, aragonite, and vaterite, as indicated.

FIG. 2A is a photograph of a biocompatible bioplastic aragonite composition containing 40% aragonite formed by three-dimensional (3D) printing of extruded filament, according to embodiments of the present disclosure.

FIG. 2B is a photograph of a biocompatible bioplastic aragonite composition containing 40% aragonite formed by three-dimensional (3D) printing of extruded filament, according to embodiments of the present disclosure.

FIGS. 3A-3D show the results of lyophilized adenoviral vectors (Ad5) of known viral titer loaded into capsules with either Aragonite or Lactose. The infectious units/gram (no acid (FIG. 3A) or with acid (FIG. 3C)) or the percentage of virus recovery (with no acid (FIG. 3B) or with acid (FIG. 3D) using the following aragonite or lactose compounding agents is shown: Aragonite coated (C) 1 (0.357 g dry, powder, pH 8.81); Aragonite (C) 5 (0.425 g dry, powder, pH 8.84); Lactose non-coated (NC) 4 (0.8023 g, liquid, pH 2.14); Aragonite (NC) 6 (0.545 g, paste, pH 7.78); Aragonite (NC) 7 (0.629 g, paste, pH 7.79) and Aragonite (NC) 8 (0.801 g, paste, pH 7.00). Samples compounded with aragonite were maintained at >/= 7 pH when exposed to acid. Each bar is one capsule.

### DETAILED DESCRIPTION

The following passages describe different aspects in greater detail. Each aspect may be combined with any other aspect or aspects unless clearly indicated to the contrary. In particular, any feature indicated as being preferred or advantageous may be combined with any other feature of features indicated as being preferred or advantageous.

Certain features of the present disclosure are described in the context of separate embodiments, but may also be provided in combination in a single embodiment. Conversely, various features of the present disclosure, which are described in the context of a single embodiment may also be provided separately or in any suitable sub-combination. All combinations of the disclosed embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed.

The present disclosure is not limited to particular embodiments described herein. Terminology used herein for describing particular embodiments only is not intended to be limiting. Publications discussed herein are provided solely for their disclosure prior to the filing date of the present application. Nothing herein is an admission that the present disclosure is not entitled to antedate such publication by virtue of prior disclosure. Further, the publication dates provided may be different from actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by an ordinary vaccine scientist. Although any methods and materials similar or equivalent to those described herein can also be used in the practice or testing of the vaccines disclosed herein, the preferred methods and materials are now described.

As disclosed herein, vaccine compositions are provided in a form that is stable and easy to administer. More specifically, freeze-drying a recombinant virus-based vaccine, and combining the vaccine with an appropriate stabilizing compound produces a composition that is stable and can be packaged for stable storage, transport, and easy administration. Thus, a composition of the present disclosure can be produced by lyophilizing an adenovirus vector and combining the adenovirus vector with a carbonite mineral, such as aragonite. Accordingly, a composition of the present disclosure generally comprises a lyophilized adenovirus vector and a carbonite mineral, such as aragonite.

Subject matter further includes compositions of and methods for producing a dosage form of a vaccine using aragonite to form a solid dosage form (e.g., powder, tablet, or capsule) that is stable during storage, easily administered (e.g., self-administered), and readily disintegrates upon mucosal administration (e.g., sublingual or buccal administration) or in an aqueous solution for consumption.

In particular, the inventive subject matter is directed to an aragonite composition made of a plurality of aragonite particles, wherein the aragonite particles are capable of being loaded with a vaccine composition rendering a solid dosage vaccine in the form of a powder, tablet, or capsule. In exemplary embodiments, the vaccine composition immunizes against a coronavirus. More specifically, the vaccine composition is a recombinant viral expression construct for expressing a corresponding antigen to the relevant infection/disease. Preferably, the recombinant construct is an adenovirus construct expressing an antigenic coronavirus protein or protein fragment.

Notably, as further detailed herein, the use of aragonite in the presently contemplated solid dosage form allows for cost effective manufacturing and easy administration of a stable vaccine composition. As such, the presently contemplated vaccine in powder, tablet, or capsule form can be mass produced and easily transported. Furthermore, the rapid disintegration (e.g., 30 seconds or less) of the solid dosage form allows for self-administration. For example, a vaccine in powder form can be packaged in individual dose packaging. Exemplary packaging of the dose form powder is similar to the packaging around a TWININGS® teabag. The vaccine powder may be opened and dissolved in water or a suitable liquid beverage for consumption (e.g., from a drinking vessel or dropper) thereby orally administering the vaccine released in the liquid. Additionally, for a vaccine in any of powder, tablet, or capsule form, given the disintegration of the dosage form upon contact with an aqueous fluid, upon sublingual or buccal administration, a person’s saliva will disintegrate the dosage form thereby releasing the vaccine composition into the oral mucosa for absorption. In additional embodiments, tablets may be formed from compressed vaccine powder. Tablets may be compressed into any suitable shape, for example, round or cubed. The tablet forms may also be made with additional excipients (e.g., flavors and gelatins) to form a lozenge.

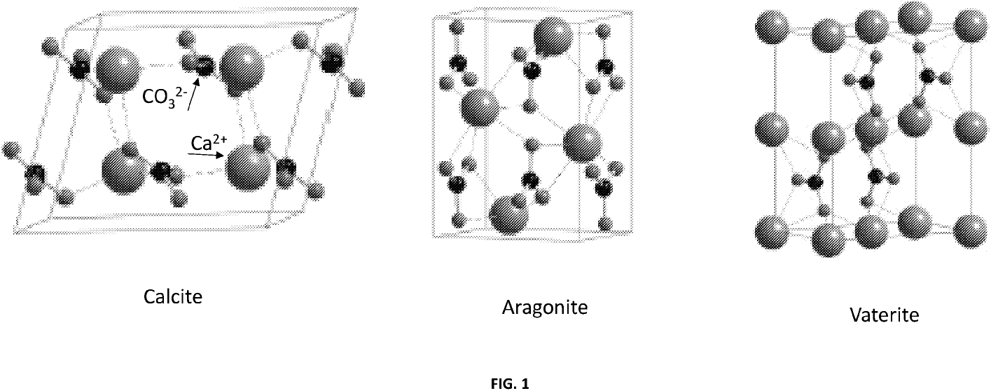

Aragonite (e.g., oolitic aragonite) is one of the purest forms of naturally precipitated calcium carbonate. With reference to FIG. 1, aragonite has a crystalline morphology of orthorhombic, bipyramidal, characteristically needle-shaped crystals, and as such is distinct from calcite and vaterite. Aragonite can be processed to recrystallize and/or reform in various shapes, such that it can be used for various purposes that take advantage of the mechanical and chemical properties of the calcium carbonate minerals. Aragonite particles as disclosed herein are solid matter having a regular (e.g., spherical, or ovoid) or irregular shape. As used herein, aragonite particles have an average particle size of between 100 nm to 1 mm. Methods for milling aragonite particles are described in U.S. 16/858,548, PCT/US20/29949, the entire contents of which are herein incorporated by reference. For example, methods for milling aragonite particles are disclosed of 2.5 to 3.5 micron size with a clean top size. A clean top size means that very few particles are larger than the 3.5 micron size when produced using the disclosed milling method with a classifier set at 2.5 to 3.5 micron size range. Accordingly, aragonite particles as disclosed herein using the methods of U.S. 16/858,548, PCT/US20/29949 have a cleaner top size than conventional GCC.

Aragonite’s adsorption capacity is a function of three parameters: (1) surface charge (also known as “ζ (zeta) potential”); (2) surface area/void ratio; and (3) particle solubility. By accurately measuring these three parameters, one can determine what materials will adsorb to aragonite particle surfaces under given conditions. Notably, the zeta potential of aragonite increases the stability of surfactants such as glycerol and sorbitol.

Furthermore, aragonite has a naturally high number of measurable pores in particles with diameters less than 2 nm (i.e., a high “microporosity”). See, e.g., EP 2719373. As such, the aragonite platform grips active ingredient particles strongly together allowing for the loaded aragonite to be formulated in a solid dosage form-e.g., powder, tablets, or capsules.

Advantageously, untreated aragonite has a neutral pH (7.8 to 8.2), a natural hydrophilic nature, electron charge (zeta potential), and already created nitrogenous pairing with amino acids and proteins. Without being bound by any one theory, these advantageous properties of aragonite render aragonite metastable under ambient conditions. More specifically, aragonite particles naturally include approximately 2-3% amino acid content, the majority of which are aspartic acid (approximately 25 to 30%) and glutamic acid (approximately 8 to 10%) rendering the aragonite surface hydrophilic. See, e.g., Mitterer, 1972, Geochimic et Cosmochimica Acta, 36: 1407-1422. Accordingly, in some embodiments a vaccine composition (e.g., recombinant adenovirus) is coupled directly to the natural, untreated surface of aragonite particles.

Currently, calcium carbonate utilized in the marketplace is processed as or from ground calcium carbonate (GCC), precipitated calcium carbonate (PCC) (synthesized), and/or limestone production. The product produced is a commodity grade with different attributes. To get a clean particle sized distribution (PSD) top size and low retain, most companies utilize a wet grinding process by either high solids or low solids. However, the product and these processes are neither biogenic nor environmentally favorable. As used herein, aragonite refers to naturally occurring aragonite having a crystalline morphology of orthorhombic, bipyramidal, and characteristically needle-shaped crystals that is distinct from GCC, PCC, and limestone.

One embodiment is a composition comprising, or consisting of: a lyophilized adenovirus vector; and a filler comprising, or consisting of, a carbonite mineral, wherein the adenovirus vector comprises a nucleic acid molecule encoding at least a portion of a heterologous protein. An “adenovirus vector” is an adenovirus, the genome of which lacks one or more genes necessary for adenovirus replication in an unmodified, mammalian cell. “Adenovirus” (“Ad” for short) refers to a group of non-enveloped, double stranded DNA viruses, approximately 60-110 nm in diameter from the family adenoviridae. Adenovirus vectors disclosed herein may be derived from adenoviruses in any of the four genera of Adenoviridae (e.g., Aviadenovirus, mastadenovirus, Atadenovirus and Siadenovirus), along with any of the serotypes of each species. In humans, most adenoviral infections are asymptomatic and have not been associated with neoplastic disease. The most extensively characterized Ad serotypes are serotype 2 (Ad2) and serotype 5 (Ad5). In one aspect, the adenovirus vector used herein is an Ad2 vector. In another aspect, the adenovirus vector is an Ad5 vector.

The adenoviral dsDNA genome is approximately 36 kb in length. The genome comprises two sets of genes: early region genes E1A, E1B, E2, E3, & E4, which are transcribed before DNA replication; and late region genes L1-L5, which are transcribed and expressed at high levels after the initiation of DNA replication. The early region genes are necessary for activating transcription of other viral regions, altering the host cellular environment to enhance virus replication, and replication of the viral DNA. The E1A transcription unit encodes two major E1as that are involved in transcriptional regulation of the virus. The two major E1bs are involved in stimulation of viral mRNA transport, blocking E1A-induced apoptosis and blocking host mRNA transport. The E2b gene encodes the viral polymerase and terminal protein precursor.

As used herein, “activities,” “functions,” and the like refer to an ability of a molecule to do something. For example, adenovirus E2a binds numerous cellular factors and modulates their activities, thereby driving the host cell into S-phase. Each of binding a cellular factor, modulating its activity, and driving the cell into S phase may be considered an E1a activity. As a consequence of lacking one or more activities, adenovirus vectors cannot replicate in unmodified, mammalian cells (“replication deficient”). As used herein, an “unmodified” mammalian cell lacks DNA encoding adenoviral protein. Replication deficient adenovirus vectors may replicate in a helper cell. A helper cell is a mammalian cell with DNA encoding a protein that provides—in trans—the one or more activities necessary for adenoviral replication that is missing from an adenovirus vector.

Modifications (also referred to as mutations) to the adenovirus genome that result in production of an adenovirus vector may be made at any location(s) in the genome, as long as the modification eliminates at least one function necessary for replication in an unmodified cell. A preferred modification results in elimination of a function that can be provided in trans in a helper cell. Useful modifications to the adenoviral genome include those that result in deletion of at least part—or all—of an adenovirus gene, so that the resulting adenovirus vector is unable to produce a functional protein having an activity necessary for replication. An adenovirus vector that lacks an activity associated with a protein is referred to as “null” for that protein, or activity, and may be signified as [protein-] (e.g., [E2b-]).

Exemplary adenoviral proteins necessary for replication in an unmodified, mammalian cell include, but are not limited to, E1a, E1b, E2a, and E2b. In one aspect, the adenovirus vector comprises a modification in a sequence encoding E1a. In one aspect, the adenovirus vector comprises a modification in a sequence encoding E1b. In one aspect, the adenovirus vector comprises a modification in a sequence encoding E2a. In one aspect, the adenovirus vector comprises a modification in a sequence encoding E2b. In one aspect, the adenovirus vector comprises a deletion in the E1 gene region. In one aspect, the adenovirus vector comprises a deletion in the E1a gene region. In one aspect, the adenovirus vector comprises a deletion in the E1b gene region. In one aspect, the adenovirus vector comprises a deletion in the E2 gene region. In one aspect, the adenovirus vector comprises a deletion in the E2a gene region. In one aspect, the adenovirus vector comprises a deletion in the E2b gene region. In one aspect, the adenovirus vector comprises a deletion in the E1 gene region and in the E2 gene region. In one aspect, the adenovirus vector comprises a deletion in one or more gene regions selected from the group consisting of the E1a gene region, the E1b gene region, the E2a gene region, and the E2b gene region. In one aspect, the adenovirus vector lacks one or more activities associated with E1a. In one aspect, the adenovirus vector lacks one or more activities associated with E1b. In one aspect, the adenovirus vector lacks one or more activities associated with E2a. In one aspect, the adenovirus vector lacks one or more associated with E2b. In one aspect, the adenovirus vector lacks one or more activities associated with one or more proteins selected from the group consisting of E1a, E1b, E2a, and E2b. In one aspect, the adenovirus vector is [E1a-] and/or [E1b-] and/or [E2a-] and/or [E2b-].

As used herein, “heterologous” refers to a molecule that comes from an organism different from that to which it is being referenced, or to a protein that comes from the same sort of organism as that in which it is expressed, but wherein the heterologous protein is expressed to a degree not typical to the tissue context in which it is being expressed. The molecule can be a protein or a nucleic acid sequence (i.e., RNA or DNA). For example, a heterologous nucleic acid sequence in a recombinant virus vector refers to the fact that the heterologous nucleic acid sequence comes from an organism other than the base virus used to construct the recombinant virus vector. As a further example, a heterologous nucleic acid sequence in an adenovirus vector refers to the fact that the heterologous nucleic acid sequence comes from an organism other than adenovirus. Similarly, a protein that is heterologous to adenovirus vector refers to the fact that the heterologous protein comes from an organism other than adenovirus.

In one aspect, the at least a portion of a heterologous protein is an immunogenic portion. As used herein, “immunogenic” refers to the ability of a specific protein portion to elicit an immune response to the specific protein, or to a protein comprising an amino acid sequence having a high degree of identity with the heterologous protein. According to this disclosure, amino acid sequences having a high degree of identity comprise contiguous amino acid sequences that are at least 80% identical, at least 85% identical, at least 87% identical, at least 90% identical, at least 92% identical, at least 93% identical, at least 94% identical, at least 95% identical, at least 96% identical, at least 97% identical, at least 98% identical or at least 99% identical. The heterologous protein may be selected from the group consisting of a neoepitope, a viral protein, and a bacterial protein. The heterologous protein may be from a virus selected from the group consisting of adenoviruses, herpesviruses, papilloma viruses, polyomaviruses, hepadnaviruses, parvoviruses, astroviruses, caliciviruses, picornaviruses, coronaviruses, flaviviruses, togaviruses, hepeviruses, retroviruses, orthomyxoviruses, arenaviruses, bunyaviruses, filoviruses, paramyxoviruses, rhabdoviruses, reoviruses, influenza, and poxviruses.

In certain embodiments, the protein is a coronaviral protein. Alphacoronaviruses and betacoronaviruses infect only mammals. Gammacoronaviruses and deltacoronaviruses infect birds, but some of them can also infect mammals. Alphacoronaviruses and betacoronaviruses usually cause respiratory illness in humans and gastroenteritis in animals. The highly pathogenic viruses, SARS-CoV, MERS-CoV, and SARS-CoV-2, cause severe respiratory syndrome in humans, and the other four human coronaviruses (HCoV-NL63, HCoV-229E, HCoV-OC43 and HKU1) induce mild upper respiratory diseases in immunocompetent hosts, although some of them can cause severe infections in infants, young children, and elderly individuals.

Coronaviruses are the largest single positive-strand RNA viruses with a genome of 27-32 kb. The genome is packed inside a helical capsid formed by the nucleocapsid protein (N) and further surrounded by an envelope. Associated with the viral envelope are at least three structural proteins: the membrane protein (M) and the envelope protein (E) that are involved in virus assembly, and the spike protein (S), which mediates virus entry into host cells. Some coronaviruses also encode an envelope-associated hemagglutinin-esterase protein (HE). Among these structural proteins, S forms large protrusions from the virus surface, giving coronaviruses the appearance of having crowns. In addition to mediating virus entry, S determines host range and tissue tropism. S also induces host immune responses.

Because they are external to the virus particle, the N, M, E, and S proteins are good candidates for developing an anti-coronaviral vaccine. In one aspect, the heterologous protein comes from a coronavirus selected from the group consisting of SARS-CoV, MERS-CoV, SARS-CoV-2, HCoV-NL63, HCoV-229E, and HCoV-OC43, HKU1. In one aspect, the heterologous protein comes from SARS-CoV-2. In one aspect, the heterologous protein comes from SARS. In one aspect, the heterologous protein comes from MERS.

In one aspect, the heterologous protein is N from a coronavirus selected from the group consisting of SARS-CoV, MERS-CoV, SARS-CoV-2, HCoV-NL63, HCoV-229E, HCoV-OC43, HKU1. In one aspect, the heterologous protein is SARS-CoV-2 N. In one aspect, the heterologous protein is N from SARS. In one aspect, the heterologous protein is N from MERS.

In one aspect, the heterologous protein is M from a coronavirus selected from the group consisting of SARS-CoV, MERS-CoV, SARS-CoV-2, HCoV-NL63, HCoV-229E, HCoV-OC43, HKU1. In one aspect, the heterologous protein is M from SARS-CoV-2. In one aspect, the heterologous protein is M from SARS. In one aspect, the heterologous protein is M from MERS.

In one aspect, the heterologous protein is E from a coronavirus selected from the group consisting of SARS-CoV, MERS-CoV, SARS-CoV-2, HCoV-NL63, HCoV-229E, HCoV-OC43, HKU1. In one aspect, the heterologous protein is E from SARS-CoV-2. In one aspect, the heterologous protein is E from SARS. In one aspect, the heterologous protein is E from MERS.

In one aspect, the heterologous protein is S from a coronavirus selected from the group consisting of SARS-CoV-2, MERS-CoV, SARS-CoV, HCoV-NL63, HCoV-229E, HCoV-OC43, HKU1. In one aspect, the heterologous protein is S from SARS-CoV-2. In one aspect, the heterologous protein is S from SARS. In one aspect, the heterologous protein is S from MERS.

In one aspect, the heterologous protein comprises an amino acid sequence at least 80%, at least 85%, at least 90%, at least 95%, at least 97%, or 100% identical to a sequence selected from the group consisting of SEQ ID NO: 1, SEQ ID NO:2, SEQ ID NO:3, and SEQ ID NO:4. In one aspect, the at least one heterologous protein comprises an amino acid sequence selected from the group consisting of SEQ ID NOs: 1-4. In one aspect, the at least a portion of the heterologous protein comprises at least six (6) contiguous amino acid residues from an amino acid sequence selected from the group consisting of SEQ ID NOs: 1-4.

Another example of viruses from which a heterologous protein may be obtained is influenza virus. Protective immune responses against influenza virus are primarily directed to the viral hemagglutinin (HA) protein-a glycoprotein on the viral surface responsible for interaction with host cell receptors. The influenza virus HA protein makes an attractive target against which to induce an immune response by vaccination. The heterologous protein may be from an influenza virus such as, but are not limited to, human influenza virus and avian influenza virus. The heterologous protein may be an influenza HA, an epitope thereof, an immunogenic portion thereof, or a variant thereof. Any influenza HA, epitope thereof, portion thereof, or variant thereof, can be used in adenovirus vectors of the present disclosure, as long as the HA protein, epitope thereof, portion thereof, or variant thereof induces an immune response, and preferably a protective immune response against influenza virus. Examples of useful influenza HA proteins, epitopes thereof, fragments thereof and variants thereof are disclosed in US 2010/0074916, US 2011/0171260, US 2011/0177122, & US 2014/0302079, the entire contents of which are incorporated herein by reference.

In one aspect, the heterologous protein is a therapeutic protein. Examples of therapeutic proteins include, but are not limited to an antibody, an Fc fusion proteins, an anticoagulant, a blood factor, a bone morphogenetic protein, an enzyme, a growth factor, a hormone, an interferon, an interleukin, and a thrombolytics protein.

Lyophilization is a process in which water is removed from a substance (e.g., an adenovirus vector) using freezing temperatures and low pressure. In an exemplary lyophilization the material to be lyophilized is cooled below its triple point, usually between -50° C. and -80° C. Once the material is frozen, the surrounding air pressure is reduced, and enough heat is added to sublimate the ice. In a second drying stage additional heat is added to remove unfrozen water molecules. Upon completion, the lyophilized material has less than 5%, usually less than 3%, and typically in the range of about 0.5% to about 3% residual moisture content. Methods of lyophilizing are described in US 7,888,097, the disclosure of which is incorporated herein by reference. In one aspect, lyophilized compositions of the present disclosure have less than 5%, less about than about 4%, less than about 3%, less than about 2%, or less than about 1% residual moisture. In one aspect, lyophilized compositions of the present disclosure have between about 0.5% and about 5% residual moisture. In one aspect, lyophilized compositions of the present disclosure have between about 0.5% and about 3% residual moisture. In one aspect, lyophilized compositions of the present disclosure have between about 0.5% and about 1% residual moisture. As used herein in regard to residual moisture content, “about” refers to a variation of no more than 10% in the referenced number.

As used herein, a “filler” is one or more compounds added to a composition to increase the composition’s mass or bulk. Exemplary fillers typically used for administration to humans and animals include, but are not limited to, lactose, sucrose, magnesium stearate, glucose, mannitol, starch, dextrose, maltodextrin, maltitol, plant cellulose, and the like. Because some individuals are sensitive to certain compounds a filler present in a composition of the present disclosure may lack one or more compounds selected from the group consisting of lactose, sucrose, magnesium stearate, glucose, mannitol, sorbitol, starch, dextrose, maltodextrin, maltitol, and plant cellulose. In one aspect, the composition lacks lactose. In one aspect, the composition lacks mannitol. In one aspect, the composition lacks sorbitol. In one aspect, the composition lacks starch.

In view of the sensitivity issues discussed above, carbonite minerals have desirable properties and thus make an excellent filler. Carbonite minerals contain a carbonate ion, CO₃²⁻. Examples of carbonite fillers include, but are not limited to, calcite, vaterite, aragonite, cerrusite, strontianite, witherite, and rutherfordine. In one aspect, the filler comprises-or consists of-a compound selected from the group consisting of calcite, vaterite, aragonite, cerrusite, strontianite, witherite, and rutherfordine. In one aspect, the filler comprises a carbonite mineral having an orthorhombic lattice. In one aspect, the filler comprises-or consists of-a compound selected from the group consisting of aragonite, cerrusite, strontianite, witherite, and rutherfordine. In one aspect, the filler comprises-or consists of-aragonite. In one aspect, the filler comprises-or consists of-cerrusite. In one aspect, the filler comprises-or consists of-strontianite. In one aspect, the filler comprises-or consists of-rutherfordine.

In addition to the aforementioned ingredients, a composition of the present disclosure may, but need not, comprise additional ingredients that act as, for example, a stabilizer, a sweetener, a buffer, a binder, a carrier, a diluent, an adjuvant, and a pharmaceutically active compound. Examples of additional ingredients include, but are not limited to, sodium chloride, potassium chloride, sodium citrate, sodium phosphate, sucrose, dimethlglycine, methylsulfonylmethane, and yeast lysate. In one aspect, the composition comprises one or more of a stabilizer, a sweetener, a buffer, a binder, a carrier, a diluent, an adjuvant, and a pharmaceutically active compound. In one aspect, the composition comprises one or more compound selected from the group consisting of sodium chloride, potassium chloride, sodium citrate, sodium phosphate, sucrose, dimethlglycine, methylsulfonylmethane, TriEthyl Citrate (TEC) and yeast lysate.

The present disclosure encompasses compositions formulated for easy administration to an individual. Accordingly, one embodiment of the present disclosure is a capsule comprising a composition disclosed herein. In one embodiment, the capsule contains a composition comprising-or consisting of-a lyophilized adenovirus vector and a filler comprising-or consisting of-a carbonite mineral wherein the adenovirus vector comprises a nucleic acid sequence encoding at least a portion of a heterologous protein. The capsule may be made from one or more materials including but not limited to, cellulose, gelatin, alginate. In one aspect the capsule comprises cellulose. In one aspect, the capsule comprises alginate. In one aspect the capsule comprises gelatin. In one aspect, the capsule is enteric coated.

One embodiment is a composition comprising a lyophilized adenovirus vector and a filler comprising aragonite, wherein the adenovirus vector lacks E1 and E2b activities such that it is replication deficient, wherein the adenovirus vector comprises a nucleic acid molecule encoding at least a portion of a protein from a coronavirus selected from the group consisting of SARS-CoV, MERS-CoV, SARS-CoV-2, HCoV-NL63, HCoV-229E, HCoV-OC43, HKU1, and wherein the coronavirus protein is selected from the group consisting of a coronavirus N protein, a coronavirus M protein, a coronavirus E protein, and a coronavirus S protein.

One embodiment is a method of making a composition disclosed herein. In one aspect, the method comprises: lyophilizing an adenovirus vector comprising a nucleic acid molecule encoding at least a portion of a heterologous protein; and combining the adenovirus vector with a filler comprising-or consisting of-a carbonite mineral.

In a further embodiment the composition is encapsulated within a capsule suitable for administration to an individual. In one aspect, the adenovirus vector is derived from adenoviruses in any of the four genera of the family Adenoviridae (e.g., Aviadenovirus, mastadenovirus, Atadenovirus and Siadenovirus), along with any of the serotypes of each species. In one aspect, the adenovirus vector comes from a serotype 2 adenovirus. In one aspect, the adenovirus vector comes from a serotype 5 adenovirus.

In one aspect, the lyophilized adenovirus vector has less than 5%, less about than about 4%, less than about 3%, less than about 2%, or less than about 1% residual moisture. In one aspect, the lyophilized adenovirus vector has between about 0.5% and about 5% residual moisture. In one aspect, the lyophilized adenovirus vector has between about 0.5% and about 3% residual moisture.

In one aspect, the filler includes one or more compounds selected from the group consisting of lactose, sucrose, magnesium stearate, glucose, mannitol, sorbitol, starch, dextrose, maltodextrin, maltitol, and plant cellulose. In one aspect, the filler lacks one or more compounds selected from the group consisting of lactose, sucrose, magnesium stearate, glucose, mannitol, sorbitol, starch, dextrose, maltodextrin, maltitol, and plant cellulose. In one aspect, the filler lacks lactose. In one aspect, the filler lacks mannitol. In one aspect, the filler lacks sorbitol. In one aspect, the filler lacks starch.

In one aspect, the composition comprises one or more of a stabilizer, a sweetener, a buffer, a binder, a carrier, a diluent, an adjuvant, and a pharmaceutically active compound. In one aspect, the composition comprises one or more compound selected from the group consisting of sodium chloride, potassium chloride, sodium citrate, sodium phosphate, sucrose, dimethlglycine, methylsulfonylmethane, and yeast lysate.

In one aspect, the capsule comprises one or more materials selected from the group consisting of cellulose, gelatin, and alginate. In one aspect the capsule comprises cellulose. In one aspect, the capsule comprises alginate. In one aspect the capsule comprises gelatin. In one aspect, the capsule is enteric coated.

Disclosed herein are methods of vaccinating an individual against an infectious organism. These methods comprise administering to the individual-one or more times-a composition disclosed herein, or a capsule comprising a composition disclosed herein. In one aspect, the composition comprises a lyophilized adenovirus vector and a filler comprising-or consisting of-a carbonite mineral wherein the adenovirus vector comprises a nucleic acid sequence encoding at least a portion of a heterologous protein from the infectious organism. In one aspect, the individual is at risk of being exposed to the infectious organism. Such individuals may be individuals who may be exposed to an infectious agent at some time or have been previously exposed but do not yet have symptoms of infection.

Routes and frequency of administration of the compositions, and capsules comprising compositions, described herein, as well as dosage, will vary from individual to individual, and from disease to disease, and may be readily established using standard techniques. In general, the compositions may be administered by an administration route including intravenous, oral, parenteral, intra-arterial, cutaneous, subcutaneous, intramuscular, topical, intracranial, intraorbital, ophthalmic, intravitreal, intraventricular, intracapsular, intraspinal, intracisternal, intraperitoneal, intranasal, aerosol, central nervous system (CNS) administration, and administration by suppository (for vaginal or rectal delivery). In general, capsules comprising compositions disclosed herein are administered orally.

A method of administering the composition of the present disclosure would depend on factors such as the age, weight, and physical condition of the patient being treated, and the disease or condition being treated. The skilled worker would, thus, be able to select a method of administration optimal for a patient on a case-by-case basis.

One embodiment is a kit. Kits may include, for example, adenovirus vectors of this disclosure, nucleic acid molecules for constructing adenovirus vectors of this disclosure, fillers of this disclosure, compositions of this disclosure, and/or capsules of this disclosure. Kits may also comprise associated components, such as, but not limited to, media, buffers, labels, containers, vials, syringes, and instructions for using the kit.

### Solid Dosage Form for Vaccine Composition

As disclosed herein, the dosage form (also referred to as the solid dosage form) includes aragonite with its inherently stable properties which is capable of being directly coupled with the vaccine composition. In exemplary embodiments, the contemplated dosage form includes aragonite impregnated with (i.e., coupled with) carbon dioxide (CO2) prior to the addition of the vaccine composition. See, e.g., EP 2719373 and US 2020/0155458. In additional embodiments, the contemplated dosage form includes aragonite with a biocompatible polymer and/or a disintegrating agent mixed and processed with the aragonite prior to the addition of the vaccine composition. Typically, aragonite is impregnated with CO2 and mixed with both a biocompatible polymer and a disintegrating agent prior to the addition of the vaccine composition. More typically, aragonite is impregnated with CO2, mixed with a biocompatible polymer and a disintegrating agent, and formed (e.g., compressed) into a solid form prior to the addition of the vaccine composition. See, e.g., EP 2719373 and US 2020/0155458.

A vaccine composition is loaded on (e.g., mixed with) the contemplated solid dosage form of aragonite as disclosed herein (e.g., optionally with impregnated CO2, a biocompatible polymer, and/or a disintegrating agent). The contemplated solid dosage form may be compressed before or after loading of the vaccine composition. Typically, the solid dosage form is compressed (e.g., compacted) before the vaccine composition is loaded thereon. In exemplary embodiments, the solid dosage form compressed (i.e., compacted) with the vaccine composition loaded thereon is milled to form powder, tablets, or capsules.

With respect to compacting the solid dosage form before or after loading of the vaccine composition is carried out using a compressive force of between 5 to 500 kN. Preferably, compaction of the solid dosage form before or after loading of the vaccine composition is carried out using a compressive force of between 6 to 300 kN, and most preferably of between 8 to 200 kN. More preferably, compaction of the solid dosage form before or after loading of the vaccine composition is carried out using a compressive force of between 8 to 100 kN, 8 to 50 kN, or 8 to 28 kN.

In exemplary embodiments, the CO2-coupled aragonite is mixed with at least one biocompatible polymer. Typically, the weight ratio of CO2-coupled aragonite to the biocompatible polymer is from about 95:5 to 5:95. In additional embodiments, the biocompatible polymer is a hot melt extruded biocompatible polymer. Exemplary biocompatible polymers include polylactic acid (PLA), polyethylene, polystyrene, polyvinylchloride, polyamide 66 (nylon), polycaprolactame, polycaprolactone, acrylic polymers, acrylonitrile butadiene styrene, polybenzimidazole, polycarbonate, polyphenylene oxide/sulfide, polypopylene, teflon, polylactic acid, aliphatic polyester such as polyhydroxybutyrate, poly-3-hydroxybutyrate (P3HB), polyhydroxyvalerate, polyhydroxybutyrate-polyhydroxyvalerate copolymer, poly(3-hydroxybutyrate-co-3-hydroxyvalerate), polyglyconate, poly(dioxanone) and mixtures thereof. Preferably, the biocompatible polymer resin is PLA. Preferably, the biocompatible polymer is Eudragit L30 D-55 (Evonik).

In specific embodiments, the weight ratio of the plurality of aragonite particles (i.e., the aragonite composition with or without carbon dioxide) to the biocompatible polymer is of from about 95:5 to 5:95. Preferably, the weight ratio of the plurality of aragonite particles to the biocompatible polymer is of from about 80:20 to 20:80, more preferably from 70:30 to 30:70 and most preferably from 60:40 to 40:60. For example, the weight ratio of the plurality of aragonite particles to the biocompatible polymer is of from about 50:50.

In additional embodiments, the CO2-coupled aragonite and biocompatible polymer also includes a disintegrating agent mixed (e.g., processed) therein. Examples of suitable disintegrating agents include starches (e.g., pea starch), modified cellulose gums, insoluble cross-linked polyvinylpyrrolidones, starch glycolates, micro crystalline cellulose, pregelatinized starch, sodium carboxymethyl starch, low-substituted hydroxypropyl cellulose, homopolymers of N-vinyl-2-pyrrolidone, alkyl-, hydroxyalkyl-, carboxyalkyl-cellulose esters, alginates, and microcrystalline cellulose and its polymorphic forms.

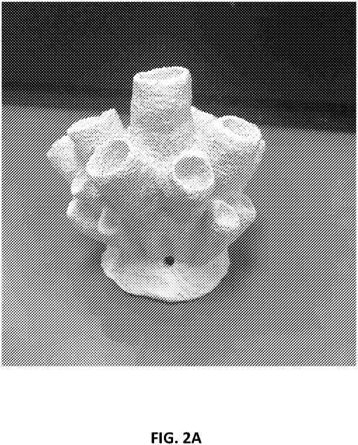

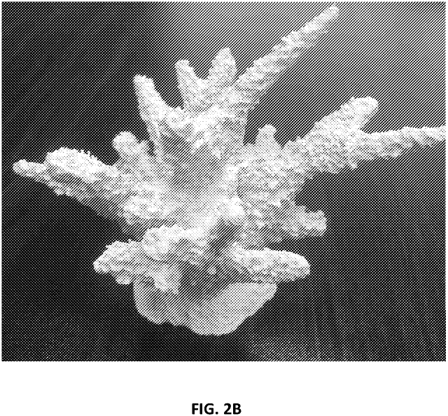

Mixing of the plurality of aragonite particles and the biocompatible polymer and optionally the disintegrating agent is carried out using any conventional hot melt extrusion method. For example, the hot melt extrusion may be carried out with a twin screw hot melt extruder with perforated die (e.g., Three-Tec, ZE9 20602, Switzerland). Compounding and extruding methods for aragonite and bioplastic compositions including filament production are described, e.g., PCT/US20/45451, the entire content of which is herein incorporated by reference. Additionally, the extruded filament composition made of a bioplastic, aragonite, and optionally the disintegrating agent, may be formed into a useful or suitable shape using 3D printing. With reference to FIGS. 2A and 2B, exemplary bioplastic aragonite (with 40% aragonite) compositions were compounded and extruded to make filaments which were then processed using 3D printing to form the aragonite structures as shown.

Additional excipients may also be added to the solid dosage form as determined by the manufacturing and packaging needs. Additional excipients may include ion exchange resins, gums, chitin, chitosan, clays, gellan gum, crosslinked polacrillin copolymers, agar, gelatine, dextrines, acrylic acid polymers, carboxymethylcellulose sodium/calcium, hydroxpropyl methyl cellulose phthalate, shellac or mixtures thereof, lubricants, inner-phase lubricants, outer-phase lubricants, impact modifiers, plasticizers, waxes, stabilizers, pigments, coloring agents, scenting agents, taste masking agents, flavoring agents, sweeteners, mouth-feel improvers, binders, diluents, film forming agents, adhesives, buffers, adsorbents, odor-masking agents and mixtures thereof.

Notably, the contemplated solid dosage form is loaded with a vaccine composition for oral, sublingual, or buccal administration. Loading or mixing of the vaccine composition to the solid dosage form (e.g., the CO2-coupled aragonite already mixed with a biocompatible polymer and a disintegrating agent) may be carried by any conventional methodology. For example, the vaccine composition may be loaded onto the solid dosage form in a mixer (e.g., tumbling mixer) or a blender.

In exemplary embodiments, contemplated solid dosage form is loaded with a vaccine composition for inducing immunity against a virus. As will be readily appreciated by the skilled artisan, there is a large variety of vaccine types depending on the disease/infection (e.g., the virus) to be immunized against. For example, where the pathogenic virus is a coronavirus (e.g., SARS-CoV, MERS-CoV, SARS-CoV-2, as well as human coronavirus NL63/HCoV-NL63), the vaccine may be a recombinant expression vector encoding all or part of the coronavirus ACE2 protein. On the other hand, where the pathogenic virus is a polio virus, the vaccine may be a recombinant expression vector encoding all or part of the CEA protein. In yet another example, where the pathogenic virus is an HIV virus, the vaccine may be a recombinant expression vector encoding all or part of gp120. Similar considerations of course apply to all other types of pathogenic viruses (e.g., influenza virus, rhinovirus, enterovirus, echovirus, herpes virus, etc.).

In exemplary embodiments, the vaccine composition is a SARS-CoV2 vaccine (e.g., an adenovirus construct) includes a soluble ACE2 protein coupled to an immunoglobulin Fc portion, forming an ACE2-Fc hybrid construct that may also include a J-chain portion, as disclosed in U.S. 16/880,804 and U.S. 63/016,048, the entire contents of both of which are herein incorporated by reference. In other exemplary embodiments, the SARS-CoV2 vaccine (e.g., an adenovirus construct) includes a mutant variant of a recombinant soluble ACE2 protein (e.g., SEQ ID NO:6), wherein the mutant variant has at least one mutated amino acid residue (e.g., by substitution) that imparts an increased binding affinity of the ACE2 protein for the RBD protein domain of the SARS-CoV2 spike protein as disclosed in U.S. 63/022,146, the entire content of which is herein incorporated by reference. In another exemplary embodiment, the SARS-CoV2 vaccine (e.g., an adenovirus construct) includes a CoV2 nucleocapsid protein or a CoV2 spike protein fused to an endosomal targeting sequence (N-ETSD), as disclosed in U.S. 16/883,263 and U.S. 63/009,960, the entire contents of both of which are herein incorporated by reference.

Preferably, the contemplated dosage form is loaded with a vaccine composition comprising a recombinant expression vector (e.g., an adenovirus) encoding a recombinant ACE2 protein as disclosed, for example, in U.S. 16/880,804, the entire contents of which are herein incorporated by reference. In typical embodiments, the vaccine composition is a recombinant human ACE2 protein having at least 85%, at least 90%, or at least 95% sequence identity to SEQ ID NO:5.

In additional or alternative embodiments, the contemplated dosage form is loaded with a vaccine composition comprising a recombinant expression vector (e.g., an adenovirus) encoding a recombinant soluble ACE2 protein (e.g., SEQ ID NO:6) or a recombinant ACE2 variant mutants including T27F, T27W, T27Y, D30E, H34E, H34F, H34K, H34M, H34W, H34Y, D38E, D38M, D38W, Q24L, D30L, H34A, and/or D355L relative to SEQ ID NO:2.

In alternative embodiments, the aragonite particle surface may be treated to modify the binding surface. For example, treatment with stearic acid (i.e., octadecanoic acid) provides for a hydrophobic surface, as disclosed in U.S. 16/858,548 and PCT/US20/29949. For protein loading, treatment of the aragonite with phosphoric acid forms lamellar structures. Additional conjugation techniques for coupling reactive groups to the amino acid surface of aragonite are known in the art as disclosed, for example, in Bioconjugate Techniques, Third Edition, Greg T. Hermanson, Academic Press, 2013.

As used herein, the singular forms “a,” “an,” and “the” include plural referents unless the context clearly dictates otherwise. For example, a nucleic acid molecule refers to one or more nucleic acid molecules. As such, the terms “a,” “an,” “one or more,” and “at least one” can be used interchangeably. Similarly, the terms “comprising,” “including,” and “having” can be used interchangeably. The claims may be drafted to exclude any optional element. As such, this statement is intended to serve as antecedent basis for use of such exclusive terminology as “solely,” “only,” and the like regarding the recitation of claim elements or use of a “negative” limitation.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE

### Example 1

This example demonstrates that non-coated aragonite formulations result in good virus recovery (pH stability and retention of solidity (i.e., paste)), while non-coated lactose formulations have less viral recovery and thus turn to liquid and become acidic.

### Capsule Formulation

Lyophilized adenoviral vector (Ad5) of known viral titer was loaded into capsules at 1 × 10⁹ infectious units (IU)/capsule. Either lactose or aragonite was loaded to a final total weight of 550 mg/capsule. Capsules were sealed under moisture-controlled conditions and optionally coated with the anionic copolymer L30 D-55 and Triethyl Citrate (TEC).

### Infectious Titer Determined by Hexon Assay

E.C7 cells are seeded into 12 well plates at approximately 5.0 × 10⁵ cells per well and incubated for at least 2 hours at 37 ± 2° C. The hAd5 construct is serially diluted in 1X DMEM (Dulbecco’s Modified Eagle Medium). Two to four hours post seeding, 100 µL per well of diluted test article is inoculated in triplicate. Adenovirus Type 5 (Ad5) Reference Material sourced from American Type Culture Collection (ATCC) is a positive control and is treated in the same manner. The negative control is 100 µL of diluent alone and is inoculated into four wells. The plates are incubated for 42 hours at 37° C. ± 2° C.

### Hexon Immunostaining

The plates are then fixed for 10 minutes with cold methanol, rinsed with 1X DPBS (Dulbecco’s phosphate-buffered saline) then assayed by Hexon immunostaining. Mouse Anti-hexon antibody solution (0.5 mL) is added to each well and incubated for 60 ± 6 minutes at 37° C. ± 2° C. Plates are washed and then 0.5 mL of Rat Anti-mouse antibody solution is added to the wells and incubated for 60 ± 6 minutes at 37° C. ± 2° C. After washing, freshly prepared DAB (diaminobenzidine) working solution is added and incubated for 10 minutes at room temperature. The DAB is aspirated and 1.0 mL of 1X PBS (phosphate-buffered saline) is added to each well.

Stained cells are visualized after 4 hours of substrate development using a light microscope at 10X objective, capturing and counting images of the entire well. The average number of positive cells/colonies per well are calculated and infectious titer is determined by the following formula: Infectious Unit/mL = (average positive cells/well) x Dilution Factor x 10.

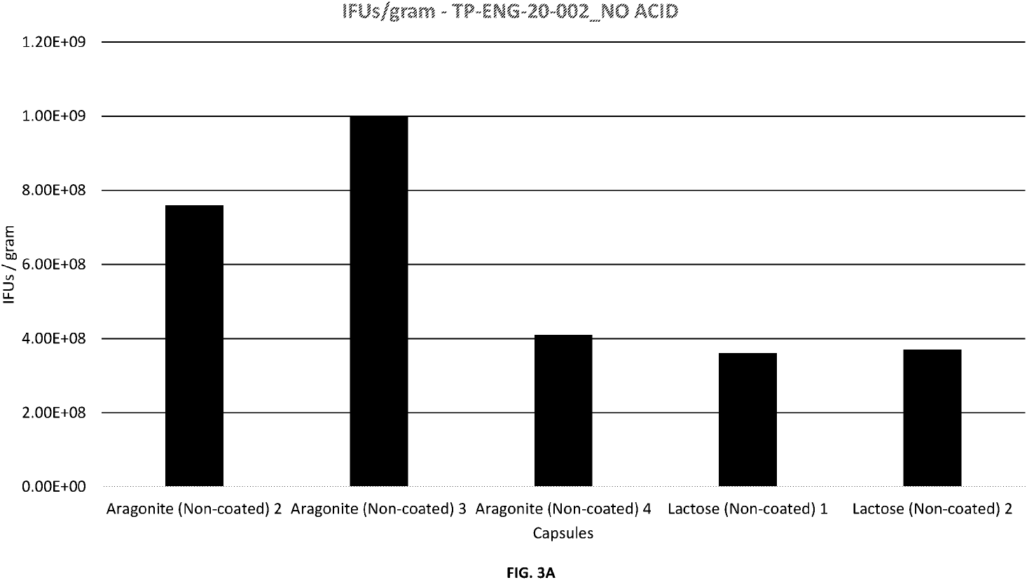

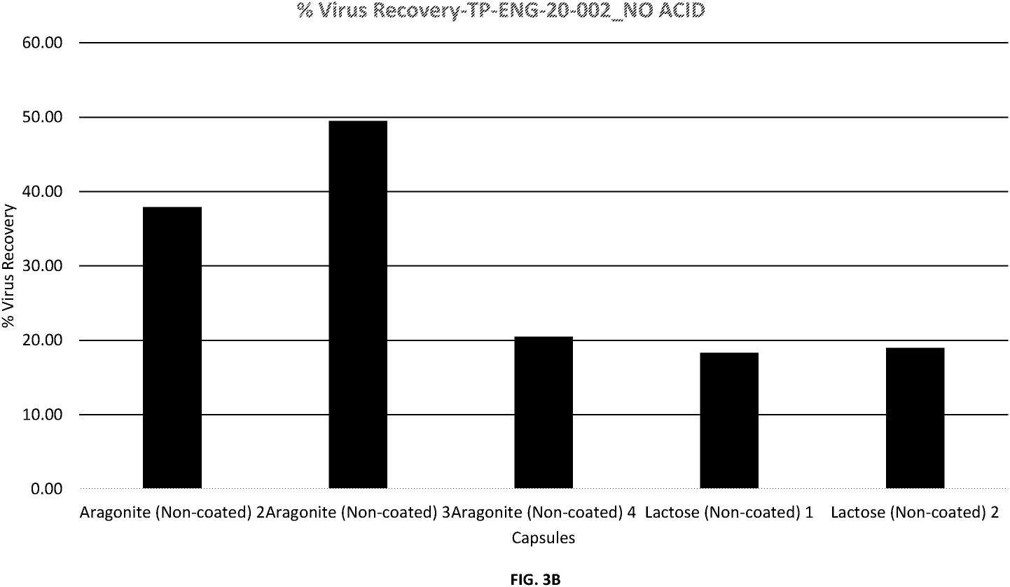

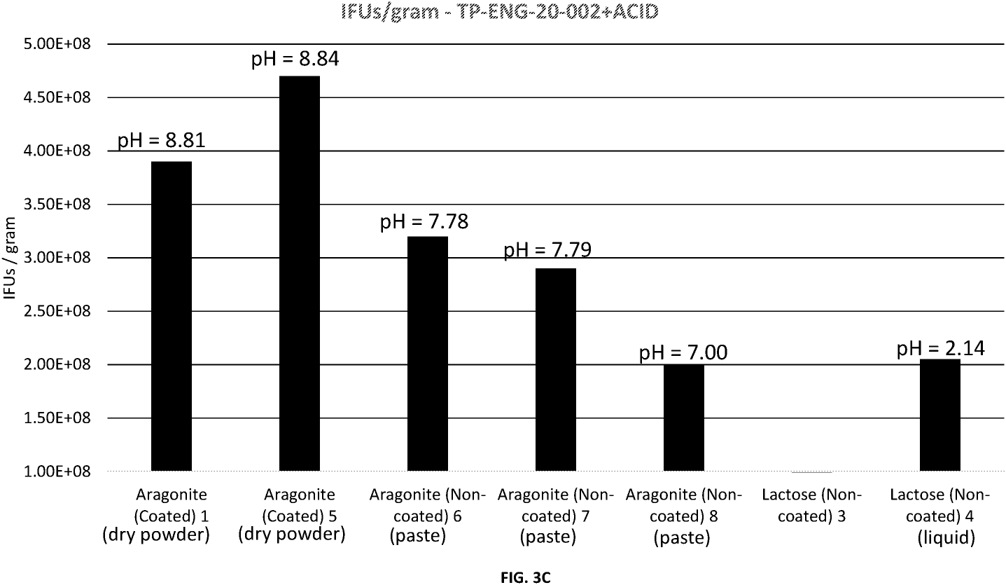

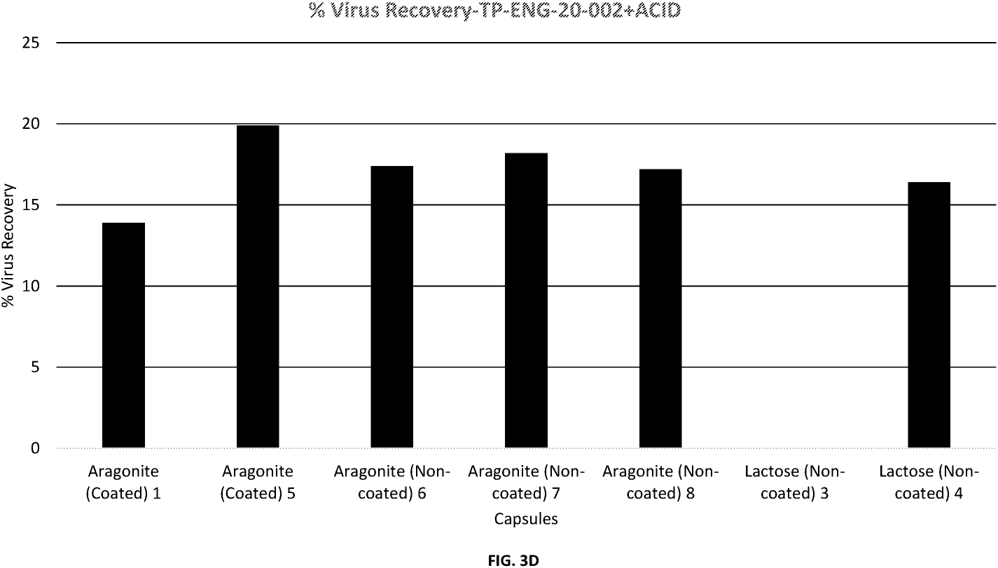

The results are shown in FIGS. 3A-3D. The data show either the infectious unit/gram (no acid (FIG. 3A) or with acid (FIG. 3C)) or the percentage of virus recovery (with no post-encapsulation acid treatment (FIG. 3B) or with post-encapsulation acid treatment (FIG. 3D) using either aragonite or lactose compounding agents. Samples compounded with aragonite were maintained at >/= 7 pH when exposed to acid. The sample mass recovered (g) is presented in Table 1 below. The end result was either a powder, liquid or paste. The pH was determined 2-minute post resuspension and read three times.

TABLE 1

Sample Mass Recovered

| Compounding Agent | Sample Mass Recovered (g) | Form | pH |
|---|---|---|---|
| Aragonite (C) 1 | 0.357 | Dry powder | 8.81 |
| Aragonite (C) 5 | 0.425 | Dry powder | 8.84 |
| Lactose (NC) 4 | 0.8023 | Liquid | 2.14 |
| Aragonite (NC) 6 | 0.545 | Paste | 7.78 |
| Aragonite (NC) 7 | 0.629 | Paste | 7.79 |
| Aragonite (NC) 8 | 0.801 | Paste | 7.00 |

Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Each publication or patent cited herein is incorporated herein by reference in its entirety.

What is claimed is:

1. A composition comprising a lyophilized adenovirus vector and a filler comprising a carbonite mineral, wherein the adenovirus vector comprises a nucleic acid molecule encoding at least a portion of a heterologous protein.

2. The composition of claim 1, wherein the adenovirus vector is derived from a type 5 adenovirus, and wherein the adenovirus has deletions in the E1, E2b, and E3 regions.

3. The composition of any one of the previous claims, wherein the heterologous protein comes from a virus.

4. The composition of claim 3, wherein the heterologous protein comes from a virus selected from the group consisting of SARS-CoV-2, MERS-CoV, SARS-CoV, HCoV-NL63, HCoV-229E, HCoV-OC43, HKU1, and influenza virus.

5. The composition of claim 4, wherein the heterologous protein comes from SARS-CoV-2.

6. The composition of claim 5, wherein the heterologous protein is a spike (S) protein, a nucleocapsid (N) protein, or a membrane (M) protein.

7. The composition of claim 6, wherein the heterologous protein is at least 80%, optionally at least 85%, optionally at least 90%, optionally at least 95%, optionally at least 97%, or even 100% identical to SEQ ID NO:1, SEQ ID NO:2, SEQ ID NO:3, or SEQ ID NO:4.

8. The composition of any one of the previous claims, wherein the adenovirus vector has between 0.5% and 5% residual moisture content.

9. The composition of claim 8, wherein the adenovirus vector has less than 5% residual moisture.

10. The composition of claim 9, wherein the adenovirus vector has less than 3% residual moisture.

11. The composition of any one of the previous claims, wherein the carbonite mineral has an orthorhombic lattice.

12. The composition of claim 11, wherein the carbonite mineral is selected from the group consisting of aragonite, cerrusite, strontianite, witherite, and rutherfordine.

13. The composition of claim 12, wherein the carbonite mineral is aragonite.

14. The composition of any one of the previous claims, wherein the filler comprises one or more compounds selected from the group consisting of lactose, sucrose, magnesium stearate, glucose, mannitol, sorbitol, starch, dextrose, maltodextrin, maltitol, and plant cellulose.

15. The composition of any one of the previous claims, wherein the filler lacks one or more compounds selected from the group consisting of lactose, sucrose, magnesium stearate, glucose, mannitol, sorbitol, starch, dextrose, maltodextrin, maltitol, and plant cellulose.

16. The composition of any one of the previous claims, wherein the composition comprises one or more compounds selected from the group consisting of sodium chloride, potassium chloride, sodium citrate, sodium phosphate, sucrose, dimethylglycine, glycine, methylsulfonylmethane, and yeast lysate.

17. A capsule comprising the composition of any one of the previous claims.

18. The capsule of claim 17, wherein the capsule is enteric coated.

19. The capsule of claim 17 or 18, wherein the capsule comprises alginate.

20. A solid dosage form for delivery of a vaccine composition by oral, sublingual, or buccal administration of the vaccine composition, the solid dosage form comprising:
an aragonite composition comprising a plurality of aragonite particles, wherein the plurality of aragonite particles are impregnated with carbon dioxide (CO2); and
a biocompatible polymer and a disintegrating agent mixed with the aragonite composition;
wherein the solid dosage form further comprises the vaccine composition, and
wherein the solid dosage form is a powder, a tablet, or a capsule.

21. The solid dosage form of claim 20, wherein the solid dosage form further comprises at least one excipient and is formulated to form a lozenge.

22. The solid dosage form of claim 20, wherein the plurality of aragonite particles have an average particle size of between 100 nm to 1 mm.

23. The solid dosage form of any of claims 20-22, wherein the biocompatible polymer is selected from polylactic acid (PLA), polyethylene, polystyrene, polyvinylchloride, polyamide 66 (nylon), polycaprolactame, polycaprolactone, acrylic polymers, acrylonitrile butadiene styrene, polybenzimidazole, polycarbonate, polyphenylene oxide/sulfide, polypopylene, teflon, polylactic acid, aliphatic polyester such as polyhydroxybutyrate, poly-3-hydroxybutyrate (P3HB), polyhydroxyvalerate, polyhydroxybutyrate-polyhydroxyvalerate copolymer, poly(3-hydroxybutyrate-co-3-hydroxyvalerate), polyglyconate, poly(dioxanone) and mixtures thereof.

24. The solid dosage form of claim 23, wherein the biocompatible polymer is PLA.

25. The solid dosage form of any of claims 20-24, wherein the disintegrating agent is selected from a starch, modified cellulose gums, insoluble cross-linked polyvinylpyrrolidones, starch glycolates, micro crystalline cellulose, pregelatinized starch, sodium carboxymethyl starch, low-substituted hydroxypropyl cellulose, homopolymers of N-vinyl-2-pyrrolidone, alkyl-, hydroxyalkyl-, carboxyalkyl-cellulose esters, alginates, and microcrystalline cellulose and its polymorphic forms.

26. The solid dosage form of claim 25, wherein the disintegrating agent is pea starch.

27. The solid dosage form of any one of claims 20-26, wherein the powder, the tablet, and the capsule disintegrate in an aqueous solution in less than 30 seconds.

28. The solid dosage form of any one of claims 20-27, wherein the vaccine composition comprises a recombinant viral expression construct encoding a viral protein or fragment thereof.

29. The solid dosage form of claim 28, wherein the viral protein or fragment thereof corresponds to a coronavirus protein or fragment thereof.

30. The solid dosage form of claim 29, wherein the coronavirus protein or fragment thereof is a SARS-CoV2 virus binding protein.

31. The solid dosage form of claim 30, wherein the SARS-CoV2 viral binding protein comprises a recombinant ACE2 protein.

32. The solid dosage form of claim 31, wherein the recombinant ACE2 protein has at least 85% sequence identity to SEQ ID NO:5.

33. The solid dosage form of claim 32, wherein the recombinant ACE2 protein comprises the sequence of SEQ ID NO:6.

34. The solid dosage form of claim 33, wherein the recombinant ACE2 protein comprises at least one mutation selected from T27F, T27W, T27Y, D30E, H34E, H34F, H34K, H34M, H34W, H34Y, D38E, D38M, D38W, Q24L, D30L, H34A, and D355L.

35. The solid dosage form of any one of claims 20-34, wherein the vaccine composition comprises an adenovirus expression construct.

36. A method of making a solid dosage form for loading a vaccine, the method comprising:
providing an aragonite composition comprising a plurality of aragonite particles impregnated with carbon dioxide (CO2);
mixing the aragonite composition with a biocompatible polymer and a disintegrating agent to form the solid dosage form; and
adding the vaccine composition to the solid dosage form.

37. The method of claim 36, wherein the mixing of the aragonite composition with the biocompatible polymer and a disintegrating agent comprises hot melt extrusion.

38. The method of any of claims 36 or 37, wherein the solid dosage form is a powder, a tablet, or a capsule.

39. The method of claim 38, wherein the solid dosage form is a tablet and wherein the method further comprises compacting the solid dosage form.

40. The method of any one of claims 36-39, wherein the aragonite composition and the biocompatible polymer are in a weight ratio of between 95:5 to 5 to 95.

41. The method of any one of claims 36-40, wherein the plurality of aragonite particles have an average particle size of between 100 nm to 1 mm.

42. The method of any of claims 36-41, wherein the biocompatible polymer is selected from polylactic acid (PLA), polyethylene, polystyrene, polyvinylchloride, polyamide 66 (nylon), polycaprolactame, polycaprolactone, acrylic polymers, acrylonitrile butadiene styrene, polybenzimidazole, polycarbonate, polyphenylene oxide/sulfide, polypopylene, teflon, polylactic acid, aliphatic polyester such as polyhydroxybutyrate, poly-3-hydroxybutyrate (P3HB), polyhydroxyvalerate, polyhydroxybutyrate-polyhydroxyvalerate copolymer, poly(3-hydroxybutyrate-co-3-hydroxyvalerate), polyglyconate, poly(dioxanone) and mixtures thereof.

43. The method of any of claims 36-42, wherein the biocompatible polymer is PLA.

44. The method of any of claims 36-43, wherein the disintegrating agent is selected from a starch, modified cellulose gums, insoluble cross-linked polyvinylpyrrolidones, starch glycolates, micro crystalline cellulose, pregelatinized starch, sodium carboxymethyl starch, low-substituted hydroxypropyl cellulose, homopolymers of N-vinyl-2-pyrrolidone, alkyl-, hydroxyalkyl-, carboxyalkyl-cellulose esters, alginates, and microcrystalline cellulose and its polymorphic forms.

45. The method of any of claims 36-44, wherein the disintegrating agent is pea starch.

46. The method of any of claims 36-45, wherein the vaccine composition comprises a recombinant viral expression construct encoding a viral protein or fragment thereof.

47. The method of claim 46, wherein the viral protein or fragment thereof corresponds to a coronavirus protein or fragment thereof.

48. The method of claim 47, wherein the coronavirus protein or fragment thereof is a SARS-CoV2 virus binding protein.

49. The method of claim 48, wherein the SARS-CoV2 viral binding protein comprises a recombinant ACE2 protein.

50. The method of claim 49, wherein the recombinant ACE2 protein has at least 85% sequence identity to SEQ ID NO: 5.

51. The method of claim 50, wherein the recombinant ACE2 protein comprises the sequence of SEQ ID NO:6.

52. The method of claim 51, wherein the recombinant ACE2 protein comprises at least one mutation selected from T27F, T27W, T27Y, D30E, H34E, H34F, H34K, H34M, H34W, H34Y, D38E, D38M, D38W, Q24L, D30L, H34A, and D355L.

53. The method of any one of claims 36-52, wherein the vaccine composition comprises an adenovirus expression construct.


SEQUENCE LISTING (6 sequences)

SEQ ID NO: 1 - protein, 419 aa, SARS-CoV-2 virus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 222 aa, SARS-CoV-2 virus
MAGSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 75 aa, SARS-CoV-2 virus
MYSFVSEETGTLIVNSVLLFLAFVVFLLVTLAILTALRLCAYCCNIVNVSLVKPSFYVYSRVKNLNSSRVPDLLV

SEQ ID NO: 4 - protein, 1273 aa, SARS-CoV-2 virus
MFVFLVLLPLVSSQCVNLTTRTQLPPAYTNSFTRGVYYPDKVFRSSVLYSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 805 aa, Homo sapiens [sequence omitted]

SEQ ID NO: 6 - protein, 594 aa, Artificial Sequence
  Soluble ACE2 mutant constructs
  MISC_FEATURE (4)..(4): Can be Gln or Leu
  MISC_FEATURE (7)..(7): Can be Thr, Phe, Trp, or Tyr
  MISC_FEATURE (10)..(10): Can be Asp, Glu, Leu
  MISC_FEATURE (14)..(14): Can be His, Glu, Phe, Lys, Met, Trp, Tyr, or Ala
  MISC_FEATURE (18)..(18): Can be Asp, Glu, Met, or Phe
  MISC_FEATURE (335)..(335): Can be Asp or Leu
IEEXAKXFLXKFNXEAEXLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGXFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA